(19) United States
(12) Patent Application Publication
(10) Pub. No.: US 2025/0295394 A1
(43) Pub. Date: Sep. 25, 2025

(54) SYSTEM AND METHOD FOR SYNCHRONIZING DATA ACQUSITION OF AN ULTRASOUND SYSTEM AND A TRACKING SYSTEM

(71) Applicant: GE Precision Healthcare LLC, Waukesha, WI (US)

(72) Inventors: Henrik JENSEN, Charlottenlund (DK); Jacob B. OLESEN, Copenhagen (DK); Fredrik GRAN, Limhamn (SE)

(21) Appl. No.: 18/615,132

(22) Filed: Mar. 25, 2024

(51) Int. Cl. A61B 8/00; A61B 8/08

(52) U.S. Cl. CPC A61B 8/54; A61B 8/4254; A61B 8/5207

(57) ABSTRACT

Various systems and methods are provided for synchronizing data acquisition of an ultrasound system and a tracking system. Ultrasound data acquisition of ultrasound data by an ultrasound system and a tracking data acquisition of tracking data by a tracking system may be synchronized. The ultrasound data and the tracking data that are temporally aligned may be received. A medical image may be generated based on the ultrasound data and the tracking data.

### TECHNICAL FIELD

The present disclosure relates, generally, to a system and a method for synchronizing an ultrasound data acquisition of ultrasound data by an ultrasound system and a tracking data acquisition of tracking data by a tracking system.

### BACKGROUND

An ultrasound system and a tracking system may be implemented together in order to permit various applications that utilize ultrasound data acquired by the ultrasound system and tracking data acquired by the tracking system. For example, an ultrasound system may acquire two-dimensional (2D) images by free-hand operation of the ultrasound probe, and a tracking system may acquire tracking data corresponding to a position and orientation of the ultrasound probe during the acquisition of the ultrasound data. An imaging system may generate three-dimensional (3D) ultrasound images by registering the 2D images using the tracking data. As another example, an ultrasound system may acquire ultrasound data of a region of interest of a subject during an interventional procedure, and a tracking system may acquire tracking data corresponding to a position and orientation of an interventional device that is navigating towards the region of interest. An imaging system may generate a medical image of the region of interest that includes a virtual representation of the interventional device using the ultrasound data and the tracking data. As another example, an ultrasound system may acquire intraoperative ultrasound data of a region of interest of a subject, and a tracking system may acquire tracking data corresponding to a position and orientation of the ultrasound probe during the acquisition of the ultrasound data. An imaging system may register the intraoperative ultrasound data with preoperative imaging data of the region of interest acquired via a different imaging modality.

Ultrasound data acquisition of the ultrasound system and tracking data acquisition of the tracking system might not be synchronized. That is, the ultrasound system may acquire ultrasound data at various time points, and the tracking system may acquire tracking data at various time points that do not temporally align with the time points of the ultrasound data acquisition. In these cases, there is a lag between the ultrasound data acquisition and the tracking data acquisition, and there might also be magnetic interference between the ultrasound data acquisition and the tracking data acquisition. Further, the ultrasound data might be correlated with the most recently acquired tracking data. This temporal misalignment between the ultrasound data acquisition and the tracking data acquisition can introduce error into the underlying application. For example, the temporal misalignment can reduce the accuracy of 3D images generated using the ultrasound data and the tracking data, reduce the accuracy of the displayed positioning and location of a virtual representation of an interventional device, reduce the accuracy of registration of intraoperative ultrasound data and preoperative imaging data, or the like.

### SUMMARY

This summary introduces concepts that are described in more detail in the detailed description. It should not be used to identify essential features of the claimed subject matter, nor to limit the scope of the claimed subject matter.

In an aspect, a method may include synchronizing an ultrasound data acquisition of ultrasound data by an ultrasound system and a tracking data acquisition of tracking data by a tracking system; receiving the ultrasound data and the tracking data that are temporally aligned, based on synchronizing the ultrasound data acquisition of the ultrasound data by the ultrasound system and the tracking data acquisition of the tracking data by the tracking system; and generating an ultrasound image based on the ultrasound data and the tracking data.

In another aspect, a device may include a memory configured to store instructions; and one or more processors configured to execute the instructions to: synchronize an ultrasound data acquisition of ultrasound data by an ultrasound system and a tracking data acquisition of tracking data by a tracking system; receive the ultrasound data and the tracking data that are temporally aligned, based on synchronizing the ultrasound data acquisition of the ultrasound data by the ultrasound system and the tracking data acquisition of the tracking data by the tracking system; and generate a medical image based on the ultrasound data and the tracking data.

In yet another aspect, a non-transitory computer-readable medium may store instructions that, when executed by one or more processors, cause the one or more processors to: synchronize an ultrasound data acquisition of ultrasound data by an ultrasound system and a tracking data acquisition of tracking data by a tracking system; receive the ultrasound data and the tracking data that are temporally aligned, based on synchronizing the ultrasound data acquisition of the ultrasound data by the ultrasound system and the tracking data acquisition of the tracking data by the tracking system; and generate a medical image based on the ultrasound data and the tracking data.

### BRIEF DESCRIPTION OF DRAWINGS

FIG. 1A is a diagram of an example system for synchronizing data acquisition of an ultrasound system and a tracking system according to an embodiment.

FIG. 1B is a diagram of an example system for synchronizing data acquisition of an ultrasound system and a tracking system according to another embodiment.

FIG. 1C is a diagram of an example system for synchronizing data acquisition of an ultrasound system and a tracking system according to another embodiment.

FIG. 2 is a diagram of example components of a synchronization system.

FIG. 3 is a diagram of example components of an ultrasound system.

FIG. 4 is a diagram of example components of a tracking system.

FIG. 5 is a flowchart of an example process for synchronizing data acquisition of an ultrasound system and a tracking system.

FIG. 6 is a diagram of an example process for synchronizing data acquisition of an ultrasound system and a tracking system.

FIG. 7 is a diagram of an example process for synchronizing data acquisition of an ultrasound system and a tracking system.

FIG. 8 is a diagram of an example process for synchronizing data acquisition of an ultrasound system and a tracking system.

FIG. 9 is a diagram of unsynchronized ultrasound data acquisition and tracking data acquisition.

FIG. 10 is a diagram of synchronized ultrasound data acquisition and tracking data acquisition.

FIG. 11 is a diagram of generating a medical image including a tracked interventional device using synchronized ultrasound data and tracking data.

FIG. 12 is a diagram of generating a medical image including ultrasound data and preoperative imaging data using synchronized ultrasound data and tracking data.

### DETAILED DESCRIPTION

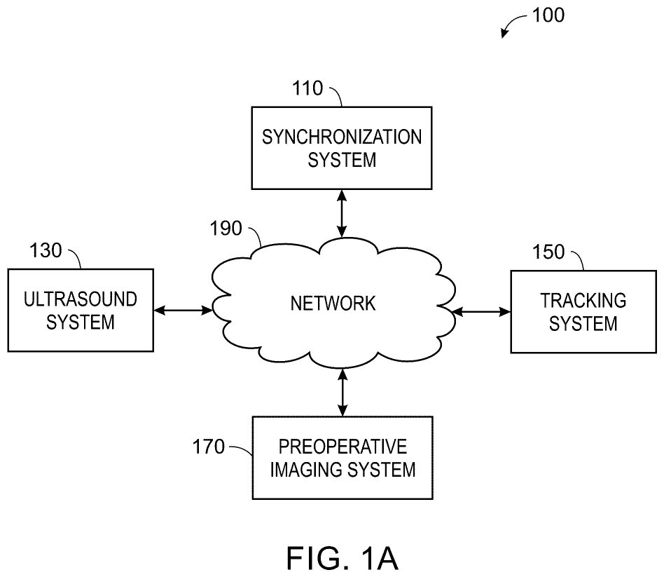

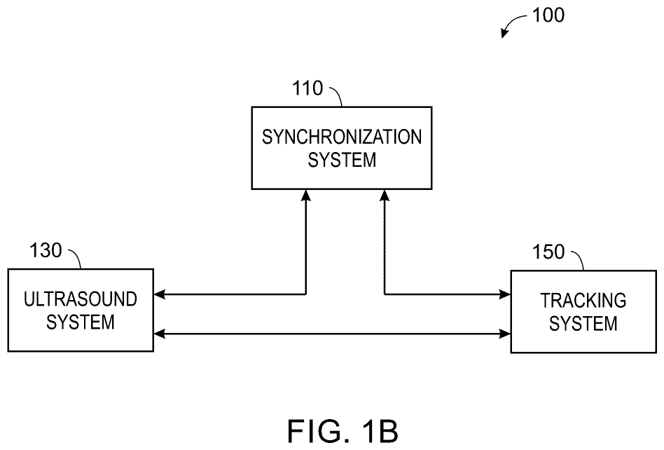

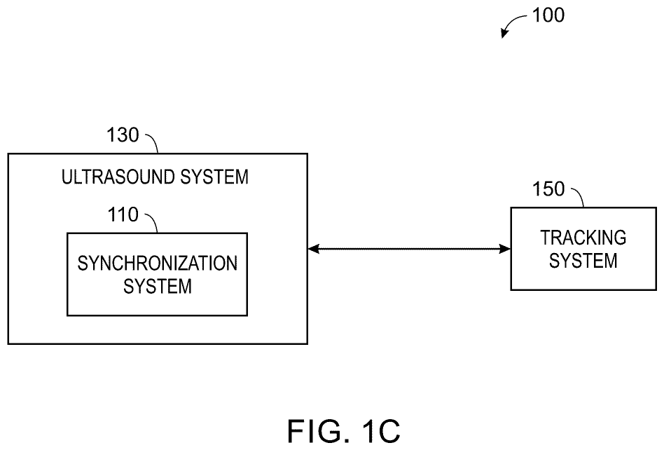

FIG. 1A is a diagram of an example system 100 for synchronizing data acquisition of an ultrasound system and a tracking system. As shown in FIG. 1A, the system 100 may include a synchronization system 110, an ultrasound system 130, a tracking system 150, a preoperative imaging system 170, and a network 190.

The synchronization system 110 may be configured to synchronize an ultrasound data acquisition of ultrasound data by the ultrasound system 130 and a tracking data acquisition of tracking data by the tracking system 150, receive the ultrasound data and the tracking data that are temporally aligned, and generate a medical based on the ultrasound data and the tracking data. For example, the synchronization system 110 may be a server, a computer, or the like.

The ultrasound system 130 may be configured to acquire ultrasound data. For example, the ultrasound system 130 may be a 2D ultrasound system, a 3D ultrasound system, a 4D ultrasound system, a Doppler ultrasound system, or the like.

The tracking system 150 may be configured to acquire tracking data. For example, the tracking system 150 may be an electromagnetic tracking system, an optical tracking system, an acoustic tracking system, an inertial tracking system, or the like.

The preoperative imaging system 170 may be configured to acquire preoperative imaging data. For example, the preoperative imaging system 170 may be a computed tomography (CT) system, a magnetic resonance imaging (MRI) system, an ultrasound system, an X-ray system, a positron emission tomography (PET) device, or the like.

The network 190 may permit communication between the synchronization system 110, the ultrasound system 130, the tracking system 150, and the preoperative imaging system 170. For example, the network 190 may be a local area network (LAN), a wide area network (WAN), a metropolitan area network (MAN), a cellular network, a private network, an ad hoc network, an intranet, the Internet, a fiber optic-based network, a wired network, a wireless network, or the like, and/or a combination of these or other types of networks.

The number and arrangement of the system 100 the ultrasound imaging system 100 are provided as an example. In practice, the system 100 may include additional systems, fewer systems, different systems, or differently arranged systems than those shown in FIG. 1. Additionally, or alternatively, a set of systems (e.g., one or more systems) of the system 100 may be integrated into a single system, and/or perform one or more functions described as being performed by another system, or set of systems, of the system 100.

FIG. 1B is a diagram of an example system for synchronizing data acquisition of an ultrasound system and a tracking system according to another embodiment. As shown in FIG. 1B, the synchronization system 110, the ultrasound system 130, and the tracking system 150 may be directly connected via a direct connection (e.g., universal serial bus (USB), RS-232, or the like).

FIG. 1C is a diagram of an example system for synchronizing data acquisition of an ultrasound system and a tracking system according to another embodiment. As shown in FIG. 1C, the ultrasound system 130 may include the synchronization system 110. Although not depicted, it should be understood that the tracking system 150 may include the synchronization system 110 according to another embodiment.

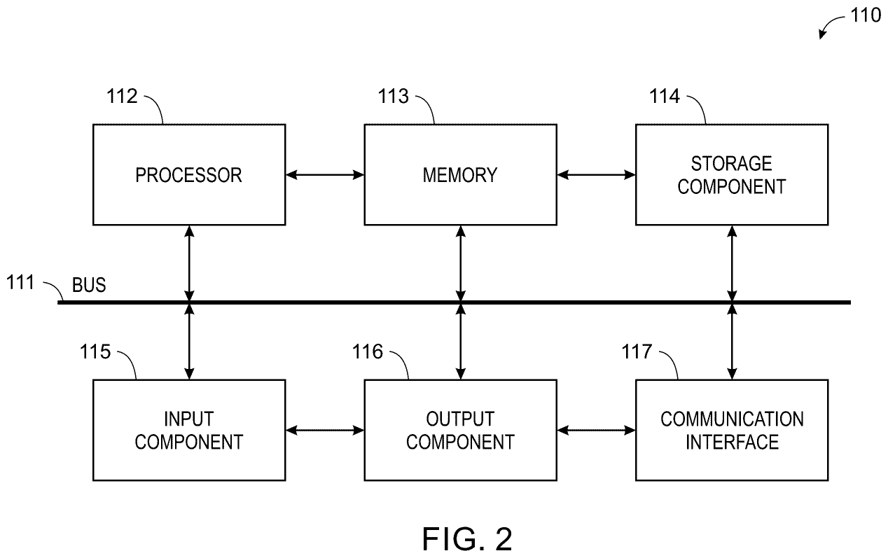

FIG. 2 is a diagram of example components of the synchronization system 110. As shown in FIG. 2, the synchronization system 110 may include a bus 111, a processor 112, a memory 113, a storage component 114, an input component 115, an output component 116, and a communication interface 117.

The bus 210 includes a component that permits communication among the components of the synchronization system 110. The processor 112 may be implemented in hardware, firmware, or a combination of hardware and software. The processor 112 may be a central processing unit (CPU), a graphics processing unit (GPU), an accelerated processing unit (APU), a microprocessor, a microcontroller, a digital signal processor (DSP), a field-programmable gate array (FPGA), an application-specific integrated circuit (ASIC), or another type of processing component.

The processor 112 may include one or more processors capable of being programmed to perform a function. The processor 112 may include one or more processors 112 configured to perform the operations described herein. For example, a single processor 112 may be configured to perform all of the operations described herein. Alternatively, multiple processors 112, collectively, may be configured to perform all of the operations described herein, and each of the multiple processors 112 may be configured to perform a subset of the operations descried herein. For example, a first processor 112 may perform a first subset of the operations described herein, a second processor 112 may be configured to perform a second subset of the operations described herein, etc.

The memory 113 may include a random access memory (RAM), a read only memory (ROM), and/or another type of dynamic or static storage device (e.g., a flash memory, a magnetic memory, and/or an optical memory) that stores information and/or instructions for use by the processor 112.

The storage component 114 may store information and/or software related to the operation and use of the synchronization system 110. For example, the storage component 114 may include a hard disk (e.g., a magnetic disk, an optical disk, a magneto-optic disk, and/or a solid state disk), a compact disc (CD), a digital versatile disc (DVD), a floppy disk, a cartridge, a magnetic tape, and/or another type of non-transitory computer-readable medium, along with a corresponding drive.

The input component 115 may include a component that permits the synchronization system 110 to receive information, such as via user input (e.g., a touch screen display, a keyboard, a keypad, a mouse, a button, a switch, a camera, and/or a microphone). Additionally, or alternatively, the input component 115 may include a sensor for sensing information (e.g., a global positioning system (GPS) component, an accelerometer, a gyroscope, and/or an actuator). The output component 116 may include a component that provides output information from the synchronization system 110 (e.g., a display, a speaker for outputting sound at the output sound level, and/or one or more light-emitting diodes (LEDs)).

The communication interface 117 may include a transceiver-like component (e.g., a transceiver and/or a separate receiver and transmitter) that enables the synchronization system 110 to communicate with other systems, such as via a wired connection, a wireless connection, or a combination of wired and wireless connections. The communication interface 117 may permit the synchronization system 110 to receive information from another system and/or provide information to another system. For example, the communication interface 117 may include an Ethernet interface, an optical interface, a coaxial interface, an infrared interface, a radio frequency (RF) interface, a universal serial bus (USB) interface, a Wi-Fi interface, a cellular network interface, or the like.

The synchronization system 110 may perform one or more processes described herein. The synchronization system 110 may perform these processes based on the processor 112 executing software instructions stored by a non-transitory computer-readable medium, such as the memory 113 and/or the storage component 114. A computer-readable medium may be defined herein as a non-transitory memory device. A memory device may include memory space within a single physical storage device or memory space spread across multiple physical storage devices.

The software instructions may be read into the memory 113 and/or the storage component 114 from another computer-readable medium or from another system via the communication interface 117. When executed, the software instructions stored in the memory 113 and/or the storage component 114 may cause the processor 112 to perform one or more processes described herein. Additionally, or alternatively, hardwired circuitry may be used in place of or in combination with software instructions to perform one or more processes described herein. Thus, implementations described herein are not limited to any specific combination of hardware circuitry and software.

The number and arrangement of the components shown in FIG. 2 are provided as an example. In practice, the synchronization system 110 may include additional components, fewer components, different components, or differently arranged components than those shown in FIG. 2. Additionally, or alternatively, a set of components (e.g., one or more components) of the synchronization system 110 may perform one or more functions described as being performed by another set of components of the synchronization system 110.

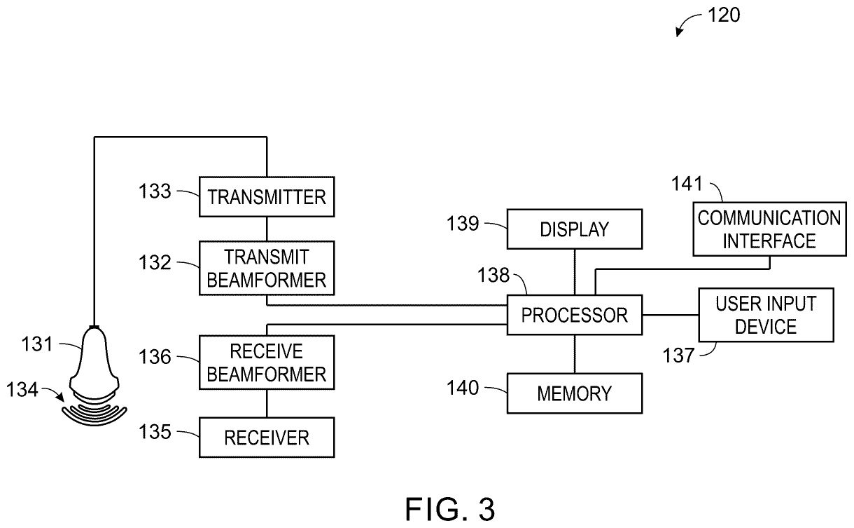

FIG. 3 is a diagram of example components of an ultrasound system 130. As shown in FIG. 1, the ultrasound system 130 may include an ultrasound probe 131, a transmit beamformer 132, a transmitter 133, elements 134, a receiver 135, a receive beamformer 136, a user input device 137, a processor 138, a display 139, a memory 140, and a communication interface 141. The foregoing components may be connected via wired or wireless connections.

The ultrasound probe 131 may be configured to acquire ultrasound data. For example, the ultrasound probe 131 may be a linear probe, a phase array probe, a curved linear probe coupled with a position tracking system, a mechanically steered linear array transducer, a phased array transducer, a curved linear array transducer, an electronically steered 2D transducer array, an electronic 3D (e3D) probe, an electronic 4d (e4D) probe, a low profile wearable patch version of any of the foregoing probes, or the like. According to an embodiment, the ultrasound probe 131 may be configured to generate ultrasound signals, emit the ultrasound signals towards a region of interest of a subject, receive echo ultrasound signals that are back-scattered from the region of interest of the subject, generate ultrasound data based on the echo ultrasound signals, and output the ultrasound data. The region of interest may be any region of the anatomy of a subject. The subject may be a person, an animal, a phantom, or the like.

The transmit beamformer 132 may be configured to apply delay times to electrical signals provided to the elements 134 to focus corresponding ultrasound signals at the region of interest. The transmitter 133 may be configured to transmit electrical signals to the elements 134 to drive the elements 134 to emit ultrasound signals towards the region of interest. The elements 134 may be configured to receive the electrical signals from the transmitter 133, convert the electrical signals into ultrasound signals, and emit the ultrasound signals towards the region of interest. The elements 134 may be configured to receive echo ultrasound signals that are back-scattered by the region of interest, convert the echo ultrasound signals into electrical signals, and provide the electrical signals to the receiver 135. The receiver 135 may be configured to receive electrical signals from the elements 134, and provide the electrical signals to the receive beamformer 136. The receive beamformer 136 may apply delay times to the electrical signals received from the elements 134.

The user input device 137 may be configured to receive a user input, and provide the user input to the processor 138. For example, the user input device 137 may be a touch screen display, a keyboard, a keypad, a mouse, a button, a switch, a microphone, or the like. Additionally, or alternatively, the user input device 137 may be configured to sense information. For example, the user input device 137 may sense information from an electro-magnetic positioning system, an inertial measurement system, an accelerometer, a gyroscope, an actuator, or the like.

The processor 138 may be configured to perform the operations as described herein. For example, the processor 138 may be a CPU, a GPU, an APU, a microprocessor, a microcontroller, a DSP, an FPGA, an ASIC, or the like. The processor 138 may be implemented in hardware, firmware, or a combination of hardware and software. The processor 138 may include one or more processors 138 configured to perform the operations described herein. For example, a single processor 138 may be configured to perform all of the operations described herein. Alternatively, multiple processors 138, collectively, may be configured to perform all of the operations described herein, and each of the multiple processors 138 may be configured to perform a subset of the operations descried herein. For example, a first processor 138 may perform a first subset of the operations described herein, a second processor 138 may be configured to perform a second subset of the operations described herein, etc.

The processor 138 may be configured to control the ultrasound probe 131 to acquire ultrasound data. The processor 138 may be configured to control which of the elements 134 are active, and control the shape of a beam emitted from the ultrasound probe 131. The processor 138 may generate ultrasound images for display. For example, the processor 138 may generate B-mode images, color Doppler images, M-mode images, color M-mode images, or the like. The ultrasound images may be 3D images, 2D images, single plane images, bi-plane images, three-plane images, multi-plane images, or the like. The ultrasound images may correspond to various anatomical planes (e.g., sagittal, coronal, and transverse) of the region of interest.

The display 139 may be configured to display information. For example, the display 139 may be a monitor, an LED display, a cathode ray tube, a projector display, a touchscreen, tablet computer, mobile phone, or the like. The display 139 may display ultrasound images based on the ultrasound data in real-time. For example, the display 139 may display the ultrasound images within one second, two seconds, five seconds, etc., of the ultrasound data being acquired by the ultrasound probe 131.

The memory 140 may be configured to store information and/or instructions for use by the processor 138. The memory 140 may be a non-transitory computer-readable medium. For example, the memory 140 may be a RAM, a ROM, a flash memory, a magnetic memory, an optical memory, or the like. The memory 140 may be configured to store instructions that, when executed by the processor 138, cause the processor 138 to perform the operations described herein.

The communication interface 141 may be configured to enable the processor 138 to communicate with other systems, such as via a wired connection, a wireless connection, or a combination of wired and wireless connections. For example, the communication interface 141 may include an Ethernet interface, an optical interface, a coaxial interface, an infrared interface, an RF interface, a USB interface, a Wi-Fi interface, a cellular network interface, or the like.

The number and arrangement of the components of the ultrasound system 130 shown in FIG. 3 are provided as an example. In practice, the ultrasound system 130 may include additional components, fewer components, different components, or differently arranged components than those shown in FIG. 3. Additionally, or alternatively, a set of components (e.g., one or more components) of the ultrasound system 130 may perform one or more functions described as being performed by another set of components of the ultrasound system 130.

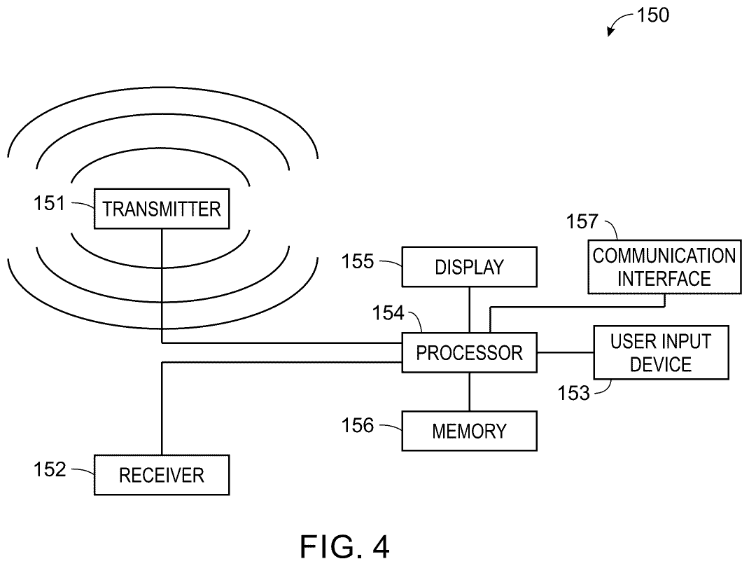

FIG. 4 is a diagram of example components of a tracking system 150. As shown in FIG. 4, the tracking system 150 may include a transmitter 151, a receiver, 152, a user input device 153, a processor 154, a display 155, a memory 156, and a communication interface 157.

The transmitter 151 may be configured to generate a magnetic field. The receiver 152 may be configured to output a signal in response to the magnetic field generated by the transmitter 151. The processor 154 may receive the output signal from the receiver 152, and acquire tracking data that identifies a position and/or an orientation of the receiver 152. According to an embodiment, the receiver 152 may be attached to the ultrasound probe 131 to track a position and/or an orientation of the ultrasound probe 131. Alternatively, the receiver 152 may be attached to an interventional device to track a position and/or an orientation of the interventional device. The interventional device may be a catheter, a needle, or the like.

The user input device 153 may be configured to receive a user input, and provide the user input to the processor 154. For example, the user input device 153 may be a touch screen display, a keyboard, a keypad, a mouse, a button, a switch, a microphone, or the like. Additionally, or alternatively, the user input device 153 may be configured to sense information. For example, the user input device 153 may sense information from an electro-magnetic positioning system, an inertial measurement system, an accelerometer, a gyroscope, an actuator, or the like.

The processor 154 may be configured to perform the operations as described herein. For example, the processor 154 may be a CPU, a GPU, an APU, a microprocessor, a microcontroller, a DSP, an FPGA, an ASIC, or the like. The processor 154 may be implemented in hardware, firmware, or a combination of hardware and software. The processor 154 may include one or more processors 154 configured to perform the operations described herein. For example, a single processor 154 may be configured to perform all of the operations described herein. Alternatively, multiple processors 154, collectively, may be configured to perform all of the operations described herein, and each of the multiple processors 154 may be configured to perform a subset of the operations descried herein. For example, a first processor 154 may perform a first subset of the operations described herein, a second processor 154 may be configured to perform a second subset of the operations described herein, etc.

The processor 154 may be configured to control the transmitter 151 to acquire tracking data. The processor 154 may be configured to control excitations of the transmitter 151 to generate a magnetic field. The processor 154 may acquire tracking data based on controlling the transmitter 151.

The display 155 may be configured to display information. For example, the display 155 may be a monitor, an LED display, a cathode ray tube, a projector display, a touchscreen, tablet computer, mobile phone, or the like. The display 155 may display the tracking data in real-time. For example, the display 155 may display the tracking data within one second, two seconds, five seconds, etc., of the tracking data being acquired.

The memory 156 may be configured to store information and/or instructions for use by the processor 154. The memory 156 may be a non-transitory computer-readable medium. For example, the memory 156 may be a RAM, a ROM, a flash memory, a magnetic memory, an optical memory, or the like. The memory 156 may be configured to store instructions that, when executed by the processor 155, cause the processor 155 to perform the operations described herein.

The communication interface 157 may be configured to enable the processor 154 to communicate with other systems, such as via a wired connection, a wireless connection, or a combination of wired and wireless connections. For example, the communication interface 157 may include an Ethernet interface, an optical interface, a coaxial interface, an infrared interface, an RF interface, a USB interface, a Wi-Fi interface, a cellular network interface, or the like.

The number and arrangement of the components of the tracking system 150 shown in FIG. 4 are provided as an example. In practice, the tracking system 150 may include additional components, fewer components, different components, or differently arranged components than those shown in FIG. 4. Additionally, or alternatively, a set of components (e.g., one or more components) of the tracking system 150 may perform one or more functions described as being performed by another set of components of the tracking system 150.

Although FIG. 4 depicts the tracking system 150 as being an electromagnetic tracking system, it should be understood that the embodiments herein are applicable to other types of tracking systems, such as optical tracking systems, acoustic tracking systems, or the like.

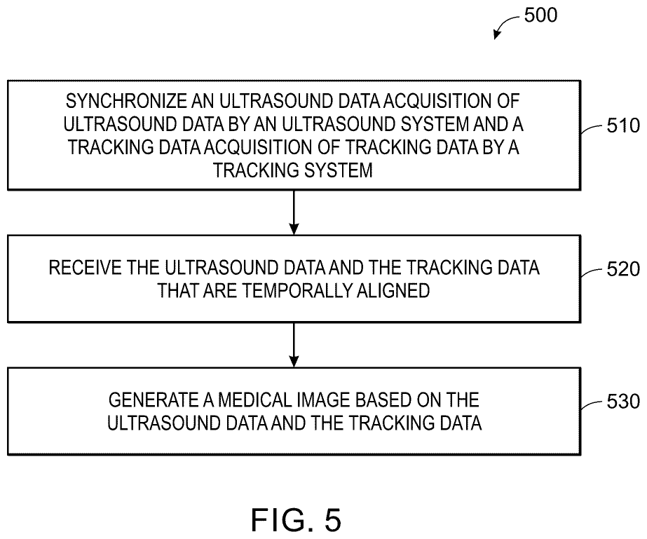

FIG. 5 is a flowchart of an example process 500 for synchronizing data acquisition of an ultrasound system and a tracking system. According to an embodiment, the process 500 may be performed by the synchronization system 110. According to another embodiment, one or more operations of the process 500 may be performed by another system, such as the ultrasound system 130 and/or the tracking system 150.

As shown in FIG. 5, the process 500 may include synchronizing an ultrasound data acquisition of ultrasound data by an ultrasound system and a tracking data acquisition of tracking data by a tracking system (operation 510), and receiving the ultrasound data and the tracking data that are temporally aligned, based on synchronizing the ultrasound data acquisition of the ultrasound data by the ultrasound system and the tracking data acquisition of the tracking data by the tracking system (operation 520).

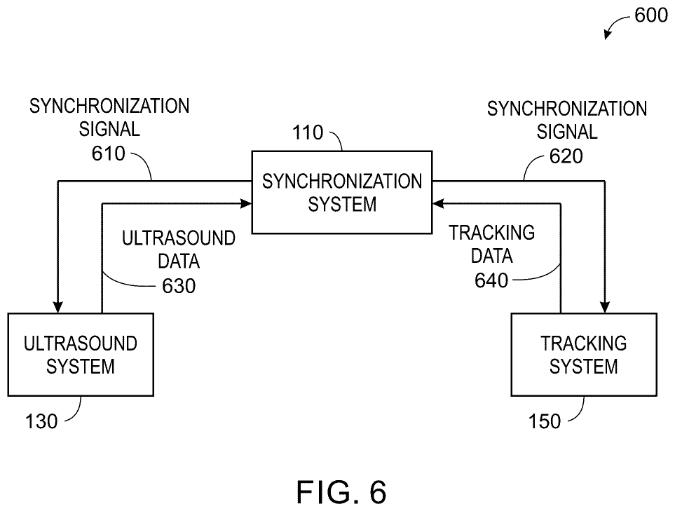

According to an embodiment, the synchronization system 110 may synchronize the ultrasound data acquisition and the tracking data acquisition by transmitting respective synchronization signals to the ultrasound system 130 and the tracking system 150. For example, as shown by reference numbers 610 and 620 in FIG. 6, which is a diagram of an example process 600 for synchronizing data acquisition of the ultrasound system 130 and the tracking system 150, the synchronization system 110 may transmit a synchronization signal to the ultrasound system 130, and may transmit a synchronization signal to the tracking system 150. The respective synchronization signals may trigger the ultrasound system 130 and the tracking system 150 to acquire ultrasound data and tracking data. As shown by reference number 630, the synchronization system 110 may receive the ultrasound data from the ultrasound system 130, and as shown by reference number 640, the synchronization system 110 may receive the tracking data from the tracking system 640.

According to an embodiment, the synchronization signal may trigger the ultrasound system 130 to perform an ultrasound data acquisition to acquire a single piece of ultrasound data. As an example, the single piece of ultrasound data may correspond to a scanline. Alternatively, the synchronization signal may trigger the ultrasound system 130 to perform a set of ultrasound data acquisitions to acquire a set of pieces of ultrasound data. The set of pieces of ultrasound data may collectively constitute an ultrasound frame. Alternatively, the set of pieces of ultrasound data may constitute a subset of an ultrasound frame. According to an embodiment, the set of pieces of ultrasound data may constitute a particular portion of an ultrasound frame. As an example, the particular portion may be a B-mode portion of the ultrasound frame. According to an embodiment, the synchronization signal may trigger the ultrasound system 130 to acquire the ultrasound data at a particular timing. For example, the synchronization signal may trigger the ultrasound system 130 to acquire ultrasound data at a particular discrete time point. Additionally, or alternatively, the synchronization signal may trigger the ultrasound system 130 to acquire ultrasound data at a particular discrete time point and at a particular acquisition rate. According to an embodiment, the processor 138 of the ultrasound system 130 may receive the synchronization signal via the communication interface 141, and control a transmission of the ultrasound probe 131 to acquire the ultrasound data.

According to an embodiment, the synchronization signal may trigger the tracking system 150 to perform a tracking data acquisition to acquire a single piece of tracking data. Alternatively, the synchronization signal may trigger the tracking system 150 to perform a set of tracking data acquisitions to acquire a set of pieces of tracking data. The set of pieces of tracking data may correspond to the set of pieces of ultrasound data that collectively constitute an ultrasound frame. Alternatively, the set of pieces of tracking data may correspond to the set of pieces of ultrasound data that constitute a subset of an ultrasound frame. According to an embodiment, the set of pieces of tracking data may correspond to the set of pieces of ultrasound data that constitute a particular portion of an ultrasound frame. As an example, the particular portion may be a B-mode portion of the ultrasound frame. According to an embodiment, the synchronization signal may trigger the tracking system 150 to acquire the tracking data at a particular timing. For example, the synchronization signal may trigger the tracking system 150 to acquire tracking data at a particular discrete time point. The particular discrete time point may be the same discrete time point as the ultrasound data acquisition of the ultrasound system 130. Additionally, or alternatively, the synchronization signal may trigger the tracking system 150 to acquire ultrasound data at a particular discrete time point and at a particular acquisition rate. According to an embodiment, the particular acquisition rate may be the same as the acquisition rate of the ultrasound system 130. According to an embodiment, the acquisition rate may be different (e.g., greater) than the acquisition rate of the ultrasound system 130. According to an embodiment, the processor 154 of the tracking system 150 may receive the synchronization signal via the communication interface 157, and control an excitation of the transmitter 151 to acquire tracking data.

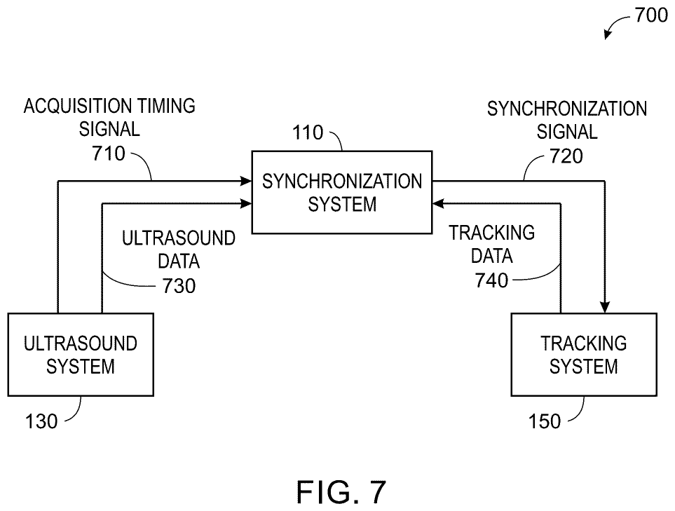

According to an embodiment, the synchronization system 110 may synchronize the ultrasound data acquisition and the tracking data acquisition by receiving an acquisition timing signal from the ultrasound system 130 and transmitting a synchronization signal to the tracking system 150. For example, as shown by reference numbers 710 and 720 in FIG. 7, which is a diagram of an example process 700 for synchronizing data acquisition of the ultrasound system 130 and the tracking system 150, the synchronization system 110 may receive an acquisition timing signal from the ultrasound system 130, and transmit a synchronization signal to the tracking system 150. The acquisition timing signal may identify a timing of an ultrasound data acquisition of ultrasound data by the ultrasound system 130, and the synchronization signal may trigger the tracking system 150 to acquire tracking data. As shown by reference number 730, the synchronization system 110 may receive the ultrasound data from the ultrasound system 130, and as shown by reference number 740, the synchronization system 110 may receive the tracking data from the tracking system 150. Although FIG. 7 depicts the synchronization system 110 receiving the acquisition timing signal from the ultrasound system 130, and transmitting the synchronization signal to the tracking system 150, the synchronization system 110 may receive an acquisition timing signal from the tracking system 150, and transmit a synchronization signal to the ultrasound system 130 according to another embodiment.

According to an embodiment, the acquisition timing signal may correspond to an ultrasound data acquisition of a single piece of ultrasound data. As an example, the single piece of ultrasound data may correspond to a scanline. Alternatively, the acquisition timing signal may correspond to a set of ultrasound data acquisitions of a set of pieces of ultrasound data. The set of pieces of ultrasound data may collectively constitute an ultrasound frame. Alternatively, the set of pieces of ultrasound data may constitute a subset of an ultrasound frame. According to an embodiment, the set of pieces of ultrasound data may constitute a particular portion of an ultrasound frame. As an example, the particular portion may be a B-mode portion of the ultrasound frame. According to an embodiment, the acquisition timing signal may identify a particular timing. For example, the acquisition timing signal may identify a particular discrete time point. Additionally, or alternatively, the acquisition timing signal may identify a particular discrete time point and at a particular acquisition rate. According to an embodiment, the processor 138 of the ultrasound system 130 may control a transmission of the ultrasound probe 131 to acquire the ultrasound data, and transmit the acquisition timing signal via the communication interface 141.

According to an embodiment, the synchronization signal may trigger the tracking system 150 to perform a tracking data acquisition to acquire a single piece of tracking data. Alternatively, the synchronization signal may trigger the tracking system 150 to perform a set of tracking data acquisitions to acquire a set of pieces of tracking data. The set of pieces of tracking data may correspond to the set of pieces of ultrasound data that collectively constitute an ultrasound frame. Alternatively, the set of pieces of tracking data may correspond to the set of pieces of ultrasound data that constitute a subset of an ultrasound frame. According to an embodiment, the set of pieces of tracking data may correspond to the set of pieces of ultrasound data that constitute a particular portion of an ultrasound frame. As an example, the particular portion may be a B-mode portion of the ultrasound frame. According to an embodiment, the synchronization signal may trigger the tracking system 150 to acquire the tracking data at a particular timing. For example, the synchronization signal may trigger the tracking system 150 to acquire tracking data at a particular discrete time point. The particular discrete time point may be the same discrete time point as the ultrasound data acquisition of the ultrasound system 130. Additionally, or alternatively, the synchronization signal may trigger the tracking system 150 to acquire ultrasound data at a particular discrete time point and at a particular acquisition rate. According to an embodiment, the particular acquisition rate may be the same as the acquisition rate of the ultrasound system 130. According to an embodiment, the acquisition rate may be different (e.g., greater) than the acquisition rate of the ultrasound system 130. According to an embodiment, the processor 154 of the tracking system 150 may receive the synchronization signal via the communication interface 157, and control an excitation of the transmitter 151 to acquire tracking data.

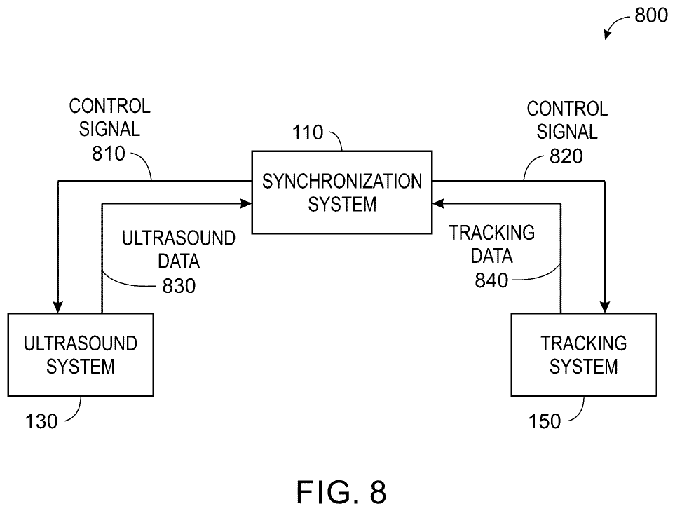

According to an embodiment, the synchronization system 110 may synchronize the ultrasound data acquisition and the tracking data acquisition by controlling the ultrasound system 130 and the tracking system 150. For example, as shown by reference numbers 810 and 820 in FIG. 8, which is a diagram of an example process 800 for synchronizing data acquisition of the ultrasound system 130 and the tracking system 150, the synchronization system 110 may transmit a control signal to the ultrasound system 130, and may transmit a control signal to the tracking system 150. The respective control signals may trigger the ultrasound system 130 and the tracking system 150 to acquire ultrasound data and tracking data. As shown by reference number 830, the synchronization system 110 may receive the ultrasound data from the ultrasound system 130, and as shown by reference number 840, the synchronization system 110 may receive the tracking data from the tracking system 150.

According to an embodiment, the control signal may control the ultrasound system 130 to perform an ultrasound data acquisition to acquire a single piece of ultrasound data. As an example, the single piece of ultrasound data may correspond to a scanline. Alternatively, the control signal may control the ultrasound system 130 to perform a set of ultrasound data acquisitions to acquire a set of pieces of ultrasound data. The set of pieces of ultrasound data may collectively constitute an ultrasound frame. Alternatively, the set of pieces of ultrasound data may constitute a subset of an ultrasound frame. According to an embodiment, the set of pieces of ultrasound data may constitute a particular portion of an ultrasound frame. As an example, the particular portion may be a B-mode portion of the ultrasound frame. According to an embodiment, the control signal may control the ultrasound system 130 to acquire the ultrasound data at a particular timing. For example, the control signal may control the ultrasound system 130 to acquire ultrasound data at a particular discrete time point. Additionally, or alternatively, the control signal may trigger the ultrasound system 130 to acquire ultrasound data at a particular discrete time point and at a particular acquisition rate. According to an embodiment, the ultrasound probe 131 may receive the control signal to acquire the ultrasound data.

According to an embodiment, the control signal may control the tracking system 150 to perform a tracking data acquisition to acquire a single piece of tracking data. Alternatively, the control signal may control the tracking system 150 to perform a set of tracking data acquisitions to acquire a set of pieces of tracking data. The set of pieces of tracking data may correspond to the set of pieces of ultrasound data that collectively constitute an ultrasound frame. Alternatively, the set of pieces of tracking data may correspond to the set of pieces of ultrasound data that constitute a subset of an ultrasound frame. According to an embodiment, the set of pieces of tracking data may correspond to the set of pieces of ultrasound data that constitute a particular portion of an ultrasound frame. As an example, the particular portion may be a B-mode portion of the ultrasound frame. According to an embodiment, the control signal may control the tracking system 150 to acquire the tracking data at a particular timing. For example, the control signal may control the tracking system 150 to acquire tracking data at a particular discrete time point. The particular discrete time point may be the same discrete time point as the ultrasound data acquisition of the ultrasound system 130. Additionally, or alternatively, the control signal may control the tracking system 150 to acquire ultrasound data at a particular discrete time point and at a particular acquisition rate. According to an embodiment, the particular acquisition rate may be the same as the acquisition rate of the ultrasound system 130. According to an embodiment, the acquisition rate may be different (e.g., greater) than the acquisition rate of the ultrasound system 130. According to an embodiment, the transmitter 151 may receive the control signal to acquire tracking data.

An ultrasound data acquisition of the ultrasound system 130 and a tracking data acquisition of the tracking system 150 may be temporally aligned. As used herein, the ultrasound data acquisition and the tracking data acquisition being “temporally aligned” may refer to a time difference between the ultrasound data acquisition of the ultrasound system 130 and the tracking data acquisition being less than a threshold amount of time. The threshold amount of time may be less than 10 milliseconds, 5 milliseconds, 1 millisecond, or the like. The ultrasound data and the tracking data may be “temporally aligned.” As used herein, the ultrasound data and the tracking data being “temporally aligned” may refer to a time difference between a time point of the ultrasound data and a time point of the tracking data being less than a threshold amount of time. The threshold amount of time may be less than 10 milliseconds, 5 milliseconds, 1 millisecond, or the like. The synchronization system 110 may receive the ultrasound data and the tracking data that are temporally aligned. The ultrasound data and the tracking data may correspond to substantially a same time point. As used herein, the ultrasound data and the tracking data “corresponding to substantially a same time point” may refer to a time difference between the ultrasound data and the tracking data being less than a threshold amount of time. The threshold amount of time may be less than 10 milliseconds, 5 milliseconds, 1 millisecond, or the like.

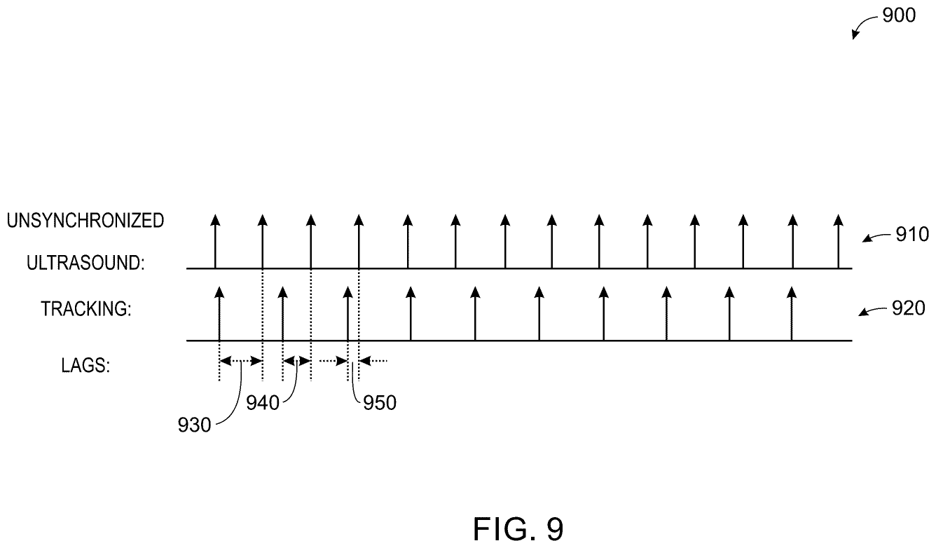

FIG. 9 is a diagram 900 of unsynchronized ultrasound data acquisition and tracking data acquisition. As shown in FIG. 9, the ultrasound system 130 may perform a set of ultrasound data acquisitions 910, and the tracking system 150 may perform a set of tracking data acquisitions 920. The ultrasound data acquisitions 910 and the tracking data acquisitions 920 are unsynchronized. An ultrasound data acquisition may be paired with the most recently performed tracking data acquisition. As shown by reference numbers 930, 940, and 950, respective lags exists between various tracking data acquisitions and ultrasound data acquisitions.

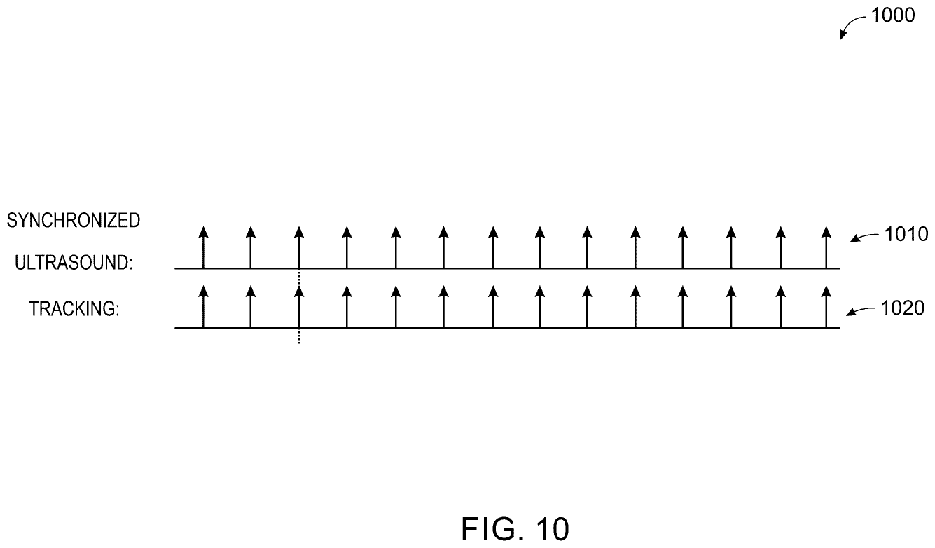

FIG. 10 is a diagram 1000 of synchronized ultrasound data acquisition and tracking data acquisition. As shown in FIG. 10, the ultrasound system 130 may perform a set of ultrasound data acquisitions 1010, and the tracking system 150 may perform a set of tracking data acquisitions 1020. The ultrasound data acquisitions 1010 and the tracking data acquisitions 1020 are synchronized. In this case, the ultrasound data acquisitions and the tracking data acquisitions are temporally aligned.

As further shown in FIG. 5, the process 500 may include generating a medical image based on the ultrasound data and the tracking data (operation 530). For example, the synchronization system 110 may generate a medical image for an application. According to an embodiment, the medical image may be a 3D ultrasound image. For example, the synchronization system may use the racking data to generate a 3D ultrasound image by registering 2D images. According to another embodiment, the medical image may be an image including a virtual representation of an interventional device (e.g., a needle, a catheter, a stent, or the like). For example, the synchronization system 110 may use the ultrasound data and the tracking data to determine a position of the interventional device, and generate a medical image including a virtual representation of the interventional device relative to a region of interest of a subject. According to another embodiment, the medical image may be an image including intraoperative imaging data and preoperative imaging data. For example, the synchronization system 110 may register intraoperative ultrasound data with preoperative imaging data of another imaging modality (e.g., CT, MRI, or the like).

By utilizing temporally aligned ultrasound data and tracking data, the embodiments herein improve the accuracy of ultrasound applications involving the utilization of ultrasound data and tracking data. For instance, the embodiments herein improve the accuracy of 3D ultrasound imaging involving the registration of 2D ultrasound images acquired via freehand movement of the ultrasound probe 131. Moreover, the embodiments herein improve the accuracy of ultrasound images that display a tracked interventional device.

The number and arrangement of the operations of the process 500 shown in FIG. 5 are provided as an example. In practice, the process 500 may include additional operations, fewer operations, different operations, differently ordered, or differently arranged operations than those shown in FIG. 5.

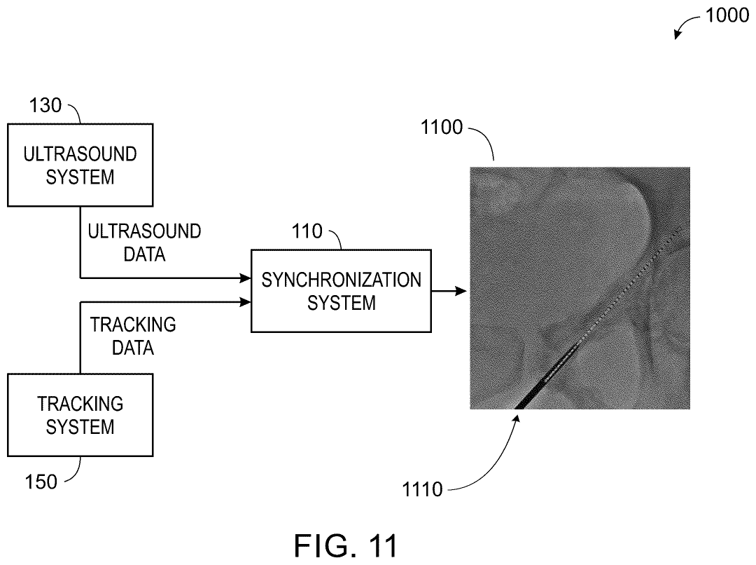

FIG. 11 is a diagram of generating a medical image including a tracked interventional device using synchronized ultrasound data and tracking data. As shown in FIG. 11, the synchronization system 110 may receive ultrasound data from the ultrasound system 130 and tracking data from the tracking system 150 that tracks an interventional device. Further, the synchronization system 110 may generate a medical image 1100 that depicts a region of interest of a subject and a virtual representation 1100 of a tracked interventional device (e.g., a needle).

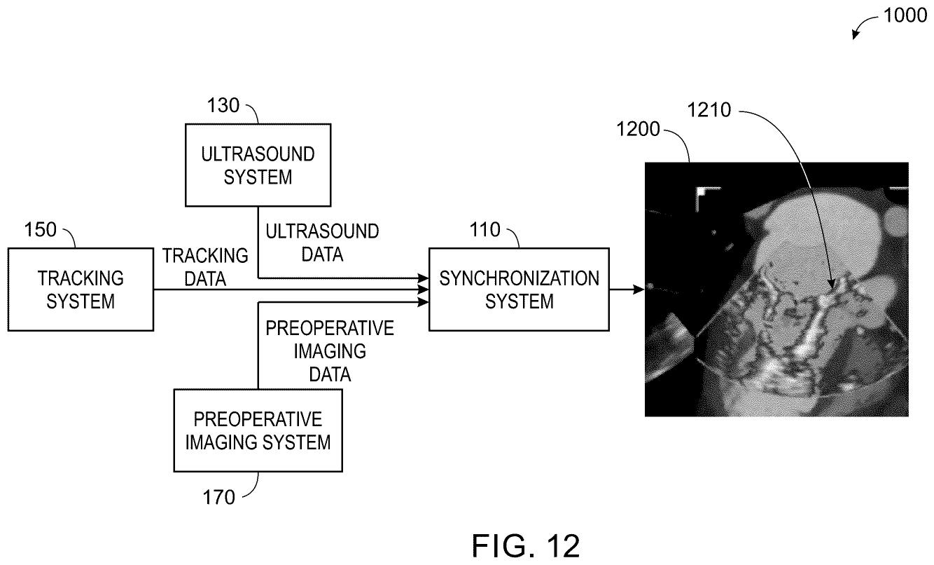

FIG. 12 is a diagram of generating a medical image including ultrasound data and preoperative imaging data using synchronized ultrasound data and tracking data. As shown in FIG. 11, the synchronization system 110 may receive ultrasound data from the ultrasound system 130, tracking data from the tracking system 150, and preoperative imaging data from the preoperative imaging system 170. Further, the synchronization system 110 may generate a medical image 1200 including the ultrasound data and the preoperative imaging data 1210 that is fused to the ultrasound data.

Embodiments of the present disclosure shown in the drawings and described above are example embodiments only and are not intended to limit the scope of the appended claims, including any equivalents as included within the scope of the claims. Various modifications are possible and will be readily apparent to the skilled person in the art. It is intended that any combination of non-mutually exclusive features described herein are within the scope of the present invention. That is, features of the described embodiments can be combined with any appropriate aspect described above and optional features of any one aspect can be combined with any other appropriate aspect. Similarly, features set forth in dependent claims can be combined with non-mutually exclusive features of other dependent claims, particularly where the dependent claims depend on the same independent claim. Single claim dependencies may have been used as practice in some jurisdictions require them, but this should not be taken to mean that the features in the dependent claims are mutually exclusive.

What is claimed is:

1. A method comprising:
synchronizing an ultrasound data acquisition of ultrasound data by an ultrasound system and a tracking data acquisition of tracking data by a tracking system;
receiving the ultrasound data and the tracking data that are temporally aligned, based on synchronizing the ultrasound data acquisition of the ultrasound data by the ultrasound system and the tracking data acquisition of the tracking data by the tracking system; and
generating a medical image based on the ultrasound data and the tracking data.

2. The method of claim 1, wherein the synchronizing the ultrasound data acquisition of the ultrasound data by the ultrasound system and the tracking data acquisition of the tracking data by the tracking system comprises transmitting a first synchronization signal to the ultrasound system that triggers the ultrasound system to acquire the ultrasound data and transmitting a second synchronization signal to the tracking system that triggers the tracking system to acquire the tracking data.

3. The method of claim 1, wherein the synchronizing the ultrasound data acquisition of the ultrasound data by the ultrasound system and the tracking data acquisition of the tracking data by the tracking system comprises receiving an acquisition timing signal from the ultrasound system and transmitting a synchronization signal to the tracking system that triggers the tracking system to acquire the tracking data.

4. The method of claim 1, wherein the synchronizing the ultrasound data acquisition of the ultrasound data by the ultrasound system and the tracking data acquisition of the tracking data by the tracking system comprises controlling the ultrasound system to acquire the ultrasound data and controlling the tracking system to acquire the tracking data.

5. The method of claim 1, wherein the ultrasound data and the tracking data correspond to substantially a same time point.

6. The method of claim 1, wherein the synchronizing the ultrasound data acquisition of the ultrasound data by the ultrasound system and the tracking data acquisition of the tracking data by the tracking system comprises synchronizing the ultrasound data acquisition of the ultrasound data by the ultrasound system and the tracking data acquisition of the tracking data for a subset of an ultrasound frame.

7. The method of claim 6, wherein the subset of the ultrasound frame corresponds to a B-mode portion of the ultrasound frame.

8. A device comprising:
a memory configured to store instructions; and
one or more processors configured to execute the instructions to:
synchronize an ultrasound data acquisition of ultrasound data by an ultrasound system and a tracking data acquisition of tracking data by a tracking system;
receive the ultrasound data and the tracking data that are temporally aligned, based on synchronizing the ultrasound data acquisition of the ultrasound data by the ultrasound system and the tracking data acquisition of the tracking data by the tracking system; and
generate a medical image based on the ultrasound data and the tracking data.

9. The device of claim 8, wherein the synchronizing the ultrasound data acquisition of the ultrasound data by the ultrasound system and the tracking data acquisition of the tracking data by the tracking system comprises transmitting a first synchronization signal to the ultrasound system that triggers the ultrasound system to acquire the ultrasound data and transmitting a second synchronization signal to the tracking system that triggers the tracking system to acquire the tracking data.

10. The device of claim 8, wherein the synchronizing the ultrasound data acquisition of the ultrasound data by the ultrasound system and the tracking data acquisition of the tracking data by the tracking system comprises receiving an acquisition timing signal from the ultrasound system and transmitting a synchronization signal to the tracking system that triggers the tracking system to acquire the tracking data.

11. The device of claim 8, wherein the synchronizing the ultrasound data acquisition of the ultrasound data by the ultrasound system and the tracking data acquisition of the tracking data by the tracking system comprises controlling the ultrasound system to acquire the ultrasound data and controlling the tracking system to acquire the tracking data.

12. The device of claim 8, wherein the ultrasound data and the tracking data correspond to substantially a same time point.

13. The device of claim 8, wherein the synchronizing the ultrasound data acquisition of the ultrasound data by the ultrasound system and the tracking data acquisition of the tracking data by the tracking system comprises synchronizing the ultrasound data acquisition of the ultrasound data by the ultrasound system and the tracking data acquisition of the tracking data for a subset of an ultrasound frame.

14. The device of claim 13, wherein the subset of the ultrasound frame corresponds to a B-mode portion of the ultrasound frame.

15. A non-transitory computer-readable medium storing instructions that, when executed by one or more processors, cause the one or more processors to:
synchronize an ultrasound data acquisition of ultrasound data by an ultrasound system and a tracking data acquisition of tracking data by a tracking system;
receive the ultrasound data and the tracking data that are temporally aligned, based on synchronizing the ultrasound data acquisition of the ultrasound data by the ultrasound system and the tracking data acquisition of the tracking data by the tracking system; and
generate a medical image based on the ultrasound data and the tracking data.

16. The non-transitory computer-readable medium of claim 15, wherein the synchronizing the ultrasound data acquisition of the ultrasound data by the ultrasound system and the tracking data acquisition of the tracking data by the tracking system comprises transmitting a first synchronization signal to the ultrasound system that triggers the ultrasound system to acquire the ultrasound data and transmitting a second synchronization signal to the tracking system that triggers the tracking system to acquire the tracking data.

17. The non-transitory computer-readable medium of claim 15, wherein the synchronizing the ultrasound data acquisition of the ultrasound data by the ultrasound system and the tracking data acquisition of the tracking data by the tracking system comprises receiving an acquisition timing signal from the ultrasound system and transmitting a synchronization signal to the tracking system that triggers the tracking system to acquire the tracking data.

18. The non-transitory computer-readable medium of claim 15, wherein the synchronizing the ultrasound data acquisition of the ultrasound data by the ultrasound system and the tracking data acquisition of the tracking data by the tracking system comprises controlling the ultrasound system to acquire the ultrasound data and controlling the tracking system to acquire the tracking data.

19. The non-transitory computer-readable medium of claim 15, wherein the ultrasound data and the tracking data correspond to substantially a same time point.

20. The non-transitory computer-readable medium of claim 15, wherein the synchronizing the ultrasound data acquisition of the ultrasound data by the ultrasound system and the tracking data acquisition of the tracking data by the tracking system comprises synchronizing the ultrasound data acquisition of the ultrasound data by the ultrasound system and the tracking data acquisition of the tracking data for a subset of an ultrasound frame.
